(19) United States
(12) Patent Application Publication
(10) Pub. No.: US 2012/0197136 A1
(43) Pub. Date: Aug. 2, 2012

(54) SELF-PUNCTURING PERCUTANEOUS OPTICAL SENSOR FOR OPTICAL SENSING OF INTRAVASCULAR FLUID

(75) Inventors: Matthew Rubin, Indianapolis, IN (US); Daniel J. Meier, Indianapolis, IN (US)

(73) Assignee: PHARMACOPHOTONICS, INC., Indianapolis, IN (US)

(21) Appl. No.: 13/318,094

(22) PCT Filed: Apr. 29, 2010
(86) PCT No.: PCT/US10/32934

§ 371 Date: Apr. 17, 2012

Related U.S. Application Data
(60) Provisional application No. 61/173,757, filed on Apr. 29, 2009.

(51) Int. Cl. A61B 5/02; A61B 6/00

(52) U.S. Cl. 600/478

(57) ABSTRACT

The present invention is directed to a self-penetrating percutaneous optical sensing device for obtaining and transmitting optical signal from intravascular fluid in a blood vessel, the device comprising: (a) an elongated hollow rigid sensor sheath 20 having a proximal end 21, a distal end 22 and a central channel extending along the sensor sheath, wherein the distal end 22 of the sensor sheath 20 is sufficiently sharpened to puncture a cutaneous barrier and the sensor sheath 20 has a sufficient length to allow the sensor sheath 20 to penetrate into intravascular space of a blood vessel; (b) a flexible optical fiber 30 having a proximal end and a distal end situated coherently within the central channel of the sensor sheath 20 wherein the sensor sheath 20 covers a portion of the distal end of the flexible optical fiber 30 and wherein the distal end of the flexible optical fiber 30 aligns with the distal end 22 of the sensor sheath 20; and (c) an optical sensor 40 connected to the distal end of the flexible optical fiber 30 wherein optical signal generated at the optical sensor 40 can be transmitted from the optical sensor 40 to the proximal end of the flexible optical fiber 30 via the flexible optical fiber 30 and wherein the optical sensor 40 has direct access to the intravascular fluid of the blood vessel.

### CROSS-REFERENCE TO RELATED APPLICATIONS

The present invention claims the benefit of U.S. Provisional Patent Application No. 61/173,757 filed Apr. 29, 2009, the contents of which are incorporated herein by reference.

### FEDERALLY SPONSORED RESEARCH OR DEVELOPMENT

N/A

### TECHNICAL FIELD

The present invention is related generally to the field of optical sensing of intravascular fluid, and specifically for a self-puncturing percutaneous optical device for obtaining and transmitting optical signal from the intravascular fluid in a blood vessel.

### BACKGROUND OF THE INVENTION

Direct optical excitation and monitoring of the intravascular space has been of commercial interest for many years. Multiple devices, such as endoscopes, cardiovascular imaging catheters, and bare imaging fibers are currently used for optical excitation and monitoring in several applications ranging from cardiovascular imaging and tissue imaging, to optical excitation of photoactive vascular components, such as fluorescently labeled carbohydrates or proteins.

As shown in the prior art (such as U.S. Pat. No. 4,782,819), conventional percutaneous optical monitoring devices comprise of an optical sheath significantly longer than their intended penetration length. This provides operators the flexibility and control to image a variety of specific substrates of interest within the body cavity during the same procedure. This flexibility and control; however, come at a significant cost of time and manual effort, requiring that operators continuously manually calibrate and adjust the physical placement of the device. As a result, operators must be highly trained, and procedures require significant financial compensation for the operators' time.

Imaging heads at the distal end of the optical sheath typically comprise a protective biologically inert coating, such as stainless steel or surgical grade plastic, an optical fiber or light pipe, and an optical focusing lens. Conventional monitoring heads are typically symmetrical and perpendicular with respect to the centerline of the optical sheath, and may also have fluid access ports allowing the operator to remove or add fluid to the location of interest. Typical Imaging heads and optical focusing lenses similarly provide the operators great control of the imaging substrate; however, they are very sensitive to change in device orientation, and also require the use of significant manual effort to properly image the intended substrate.

Conventional devices for percutaneous optical monitoring rely on the use of a separate puncture sheaths or previously exposed tissue to gain access to the patient's intravascular space. Puncture sheaths typically comprise of a separate hollow sharpened catheter made of a substantially rigid material, such as stainless steel. Typical optical imaging devices are inserted after the puncture sheath opens an access port into the vascular system. While this process is effective at gaining percutaneous access, it requires the use of multiple devices and process steps to perform.

Flexible bare optical fibers, conventionally used for detection of intravascular compounds, typically enter the vascular space and bend along the vascular wall opposite to the access port, as shown in FIG. 1. This creates what is known in the art as a walling issue, where a substantial portion of the fiber imaging substrate comprises of the vascular wall rather than the vascular fluid. Fiber walling creates noise in the optical image and decreases the fibers ability to accurately detect changes in concentrations of vascular components.

All conventional percutaneous optical imaging devices require the use of a secondary moveable device, either attached or separate, to puncture the body cavity and gain access to the intravascular space, requiring the operator to manipulate multiple devices and perform multiple process steps. In addition, while conventional optical imaging devices provide operators with significant control of the penetration depth and imaging substrate, they also require continuous manual calibration by a well trained operator. Thus, there remains a need for a simple self penetrating percutaneous optical sensing device that does not require a use of additional equipment for puncture of the vascular space and significant manual effort for the proper placement of the device. The present novel technology addresses this need.

### SUMMARY OF THE INVENTION

A first aspect of the present invention is directed to a self-penetrating percutaneous optical sensing device for obtaining and transmitting optical signal from intravascular fluid in a blood vessel, the device comprising: (a) an elongated hollow rigid sensor sheath having a proximal end, a distal end and a central channel extending along the sensor sheath, wherein the distal end of the sensor sheath is sufficiently sharpened to puncture a cutaneous barrier and the sensor sheath has a sufficient length to allow the sensor sheath to penetrate into intravascular space of a blood vessel; (b) a flexible optical fiber having a proximal end and a distal end situated coherently within the central channel of the sensor sheath wherein the sensor sheath covers a portion of the distal end of the flexible optical fiber and wherein the distal end of the flexible optical fiber aligns with the distal end of the sensor sheath; and (c) an optical sensor connected to the distal end of the flexible optical fiber wherein and optical signal generated at the optical sensor can be transmitted from the optical sensor to the proximal end of the flexible optical fiber via the flexible optical fiber and wherein the optical sensor has direct access to the intravascular fluid of the blood vessel.

The first aspect of the present invention may include one or more of the following features, alone or in any non-contradictory combination. The sensor sheath may be mechanically calibrated to a particular position and angle with respect to the first section of the sensor sheath. A portion of the proximal end of the sensor sheath may have one or more substantially planar protrusions to form stabilization platforms. The optical sensing device may further comprise a rigid sensor body covering a portion of the flexible optical fiber adjacent to the proximal end of the sensor sheath that is not covered by the sensor sheath. The rigid sensor body may be cylindrical or planar in shape. The rigid sensor body may have one or more substantially planar protrusions to form stabilization platforms. The proximal end of the flexible optical fiber may be connected to an optical detection device. The optical sensor may protrude from the sensor sheath. The optical sensor may be coated with a material containing a chemically sensitive chromophore. The optical sensing device may further comprise a centering mechanism for centering the optical sensor within the intravascular fluid of the blood vessel.

A second aspect of the present invention is directed to a self-penetrating percutaneous optical sensing device for obtaining and transmitting optical signal from intravascular fluid in a blood vessel. The optical sensing device comprises: an elongated sheath having a proximal end, a distal end and a central channel extending along the sheath, wherein the sheath has a sufficient length to allow the sheath to penetrate into intravascular space of a blood vessel; an optical fiber having a proximal end and a distal end situated coherently within the central channel of the sheath wherein the sheath covers a portion of the distal end of the optical fiber; and a centering mechanism located at the distal end of the sheath having a diameter greater than a diameter of the distal end of the sheath.

The second aspect of the invention may include one or more of the following features, alone or in any non-contradictory combination. The centering mechanism may be alternately placed in an expanded condition or a collapsed condition. The centering may be selectively elastically deformed between the expanded condition and the collapsed condition. The centering mechanism may be provided to center a tip of the optical fiber in a flow of blood. The centering device may have a central passageway through which the sheath passes. A portion of the sheath may be operably engageable with a portion of the centering mechanism and wherein relative movement between the sheath and the centering mechanism causes the centering mechanism to change from the expended condition to the collapsed condition. The centering mechanism may comprise a plurality of spokes wherein each spoke is fixed at opposing terminal ends to opposing portions of the centering mechanism. The collapsed condition may be generated by passing the sheath from a first end of the centering mechanism through the central passageway and exiting the centering mechanism at a second end of the centering mechanism. The optical sensing device may further comprise an optical sensor connected to the distal end of the optical fiber wherein an optical signal generated at the optical sensor can be transmitted from the optical sensor to the proximal end of the optical fiber via the optical fiber and wherein the optical sensor has direct access to the intravascular fluid of the blood vessel. The centering mechanism may comprise a first end segment separated from a second end segment by a midsection which expands radially outwardly upon relative movement between the first end segment and the second end segment which decreases a distance between the first and second end segments.

Further to the second aspect of the invention, a distal end of the centering mechanism may be in operative communication with the distal end of the sheath such that extension and retraction movements by the sheath are imparted to the distal end of the centering mechanism. A portion of the centering mechanism may expand radially outwardly relative to the sheath upon retraction of the distal end of the sheath towards the centering mechanism. Retraction of the distal end of the sheath may cause the distal end of the centering mechanism to enter a central portion of the centering mechanism. The distal end of the sheath may have a sharpened end for percutaneous puncture which is at least partially located within the central portion of the centering mechanism upon retraction of the distal end of the sheath. The central portion of the centering mechanism may be located radially inwardly of a plurality of spokes joining opposite the distal end of the centering mechanism with an opposing proximal end of the centering mechanism. The spokes may expand radially outwardly upon retraction of the distal end of the sheath.

One of ordinary skill on the art would readily appreciate that features from the first aspect of the invention may be combined with features from the second aspect of the invention in any reasonable, non-contradictory combination to arrive at a useful self-penetrating percutaneous optical sensing device for obtaining and transmitting optical signal from intravascular fluid in a blood vessel.

Other features and advantages of the invention will be apparent from the following specification taken in conjunction with the following drawings.

### BRIEF DESCRIPTION OF THE DRAWINGS

FIG. 1 is an illustration of an optical fiber and puncture catheter of the prior art demonstrating fiber walling against the vascular wall;

FIG. 2 is a cross-sectional diagrammatic illustration of the present invention; FIG. 2A is an embodiment wherein the sensor sheath is linear, FIG. 2B is an embodiment wherein the sensor sheath is a multi-angle sensor sheath; FIG. 2C is a further embodiment of the invention of FIG. 2B further comprising a sensor body;

FIG. 3 is an illustration of an embodiment of the present invention having a single unit sensor body and stabilization platforms, and a multi-angled sensor sheath;

FIG. 4 is an illustration of an embodiment of the present invention having a distinguished sensor body and stabilization platforms;

FIG. 5 is an illustration of an embodiment of the present invention having a substantially symmetrical sensor body and distinguished stabilization platforms;

FIG. 6 is an illustration of an embodiment of the present invention showing the optical fiber embedded within the sensor body and the sensor sheath;

FIG. 7 is an illustration of an embodiment of the present invention having a planar sensor sheath;

FIG. 8 is an illustration of an embodiment of the present invention having a sensor body without the stabilization platforms;

FIG. 9 is an illustration of an embodiment of the present invention with a multi-angle sensor sheath showing sensor body position with respect to the vascular wall;

FIG. 10 is an illustration of an embodiment of the present invention with a centering mechanism provided therewith;

FIG. 11 is an embodiment of a centering mechanism of the present invention;

FIG. 12 is an embodiment of a centering mechanism of the present invention;

FIGS. 13A through 13E show a catheter having a centering device of the present invention in use, illustrating expansion and collapsing of an elastic centering device;

FIGS. 14A through 14C show a catheter having a centering device of the present invention in use, illustrating expansion and collapsing of an alternative centering device;

FIG. 15 is an illustration of a centering mechanism of the present invention in a contracted state;

FIG. 16 is an illustration of the centering mechanism shown in FIG. 15 in a contracted state;

FIG. 17 is an illustration of a sensor sheath having side ports, apertures, openings or holes; and

FIG. 18 is an illustration of a sensor sheath having side ports, apertures, openings, or holes oriented for preferential fluid flow.

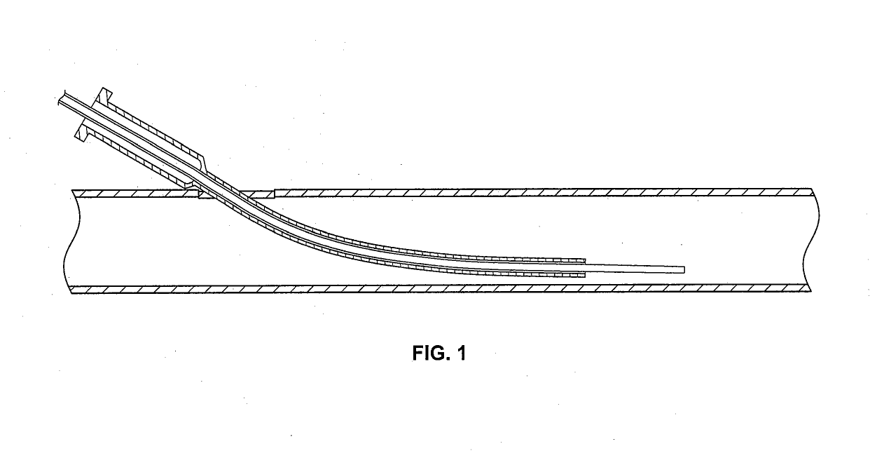

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

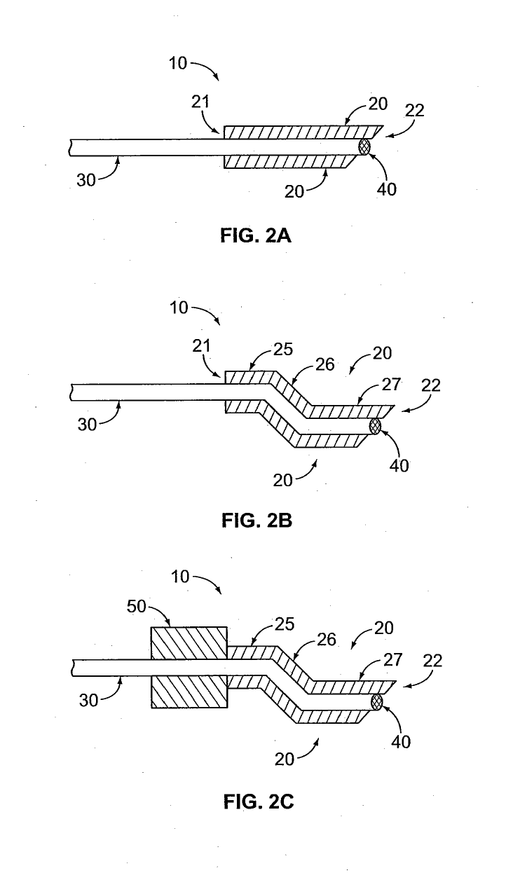

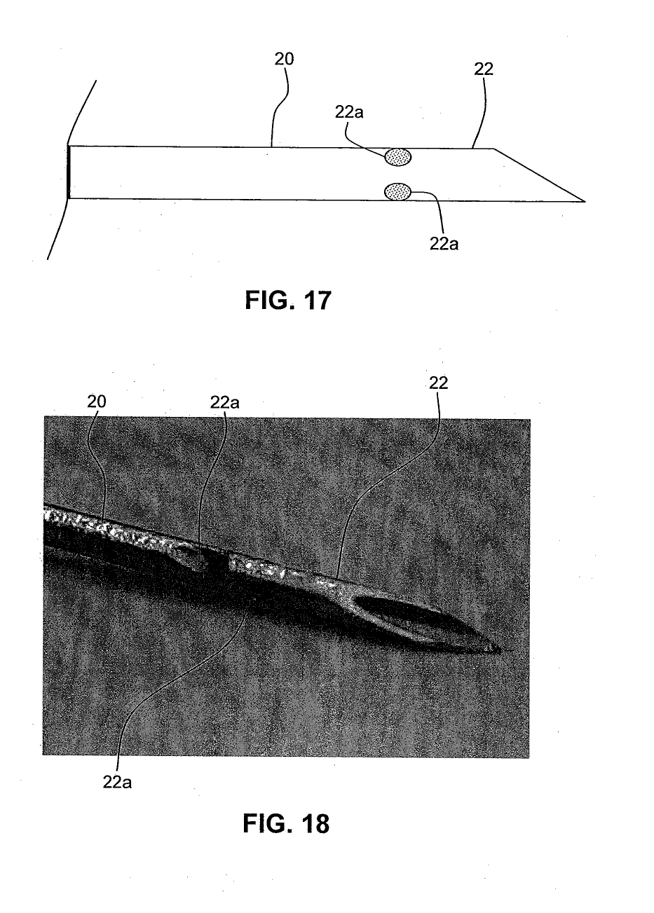

The present invention is related generally to a self-puncturing percutaneous optical sensing device for obtaining and transmitting optical signal from intravascular fluid in a blood vessel. FIG. 2 is a cross-sectional diagrammatic illustration of the present invention. The device 10 comprises: (a) an elongated hollow rigid sensor sheath 20 having a proximal end 21, a distal end 22 and a central channel extending along the sensor sheath 20, wherein the distal end 22 of the sensor sheath 20 is sufficiently sharpened to puncture a cutaneous barrier and the sensor sheath 20 has a sufficient length to allow the sensor sheath 20 to penetrate into intravascular space of a blood vessel; (b) a flexible optical fiber 30 having a proximal end and a distal end situated coherently within the central channel of the sensor sheath 20 wherein the sensor sheath 20 covers a portion of the distal end of the flexible optical fiber 30 and wherein the distal end of the flexible optical fiber 30 aligns with the distal end 22 of the sensor sheath 20; and (c) an optical sensor 40 connected to the distal end of the flexible optical fiber 30 wherein optical signal generated at the optical sensor 40 can be transmitted from the optical sensor 40 to the proximal end of the flexible optical fiber 30 via the flexible optical fiber 30 wherein the optical sensor 40 has direct access to the intravascular fluid in the blood vessel. The optical sensor 40 gains direct access to the intravascular fluid in the blood vessel through at least one opening in the sensor sheath 20. This opening may be at a location either along the sensor sheath 20 or at the distal end 22 of the sensor sheath 20. The optical sensor 40 may be flush with the sensor sheath 20. Alternatively, the optical sensor 40 may protrude or be set-in from the sensor sheath 20. In the case where the optical sensor 40 is set in the sensor sheath 20, additional holes, ports or apertures 22a in the side of the sensor near the distal end 22 may allow fluid driven by natural blood flow to enter the side of sensor sheath 20 and exit out the distal end 22, thereby further preventing walling effects while maintaining a constant supply of fresh blood in optical communication with the optical sensor 40. (See FIG. 17). This sensor sheath protrusion may be 2-20 mm in length past the optical sensor 40 and include one or multiple openings 22a that may be flush, set in, or protrude outward from the sensor sheath. The openings 22a may be oriented to encourage or enhance fluid flow from a particular direction to either enter or exit the sheath 20 via the openings 22a. (See FIG. 18).

The optical sensor 40 may further be finished with a smooth or rough surface, which may or may not include a medically acceptable lubricious hydrophilic coating, such as Hyclone Pluronic F68. In addition, the optical sensor 40 may be coated with a material containing a chemically sensitive chromophore to detect vascular the concentrations of a chemical substrate of interest. For example, a medically acceptable polymer with sufficient fluid conductivity, such as a polymer hydrogel, may contain either a bound or unbound concentration dependent monosaccharide color indicator, such as 1-(4-Boronophenylazo)-2-hydroxy-3,6-naphthalenedisulfonic acid disodium salt, as a chemically sensitive chromophore, which may be optically analyzed via the flexible optical fiber 30 to continuously monitor vascular monosaccharide concentrations.

The sensor sheath 20 can be made of a medically acceptable material with sufficient structural rigidity to puncture a cutaneous barrier. Examples of the material include but are not limited to stainless steel and surgical grade plastic.

The sensor sheath 20 may be 1 to 4 cm in length with an outer diameter of 0.5 to 1.13 mm, depending on the blood vessel of interest and the optical transmittance. A sensor sheath length longer than the minimum vascular penetration length may be used to prevent the sheath from intentionally exiting the blood vessel during normal body motion.

FIG. 2A is a cross-sectional diagrammatic illustration of the optical sensing device 10 wherein the sensor sheath 20 is linear. In a preferred embodiment, as shown in FIG. 2B, the sensor sheath 20 is a multi-angle sensor sheath. It comprises a first section 25 having a proximal end and a distal end, a second section 27 having a proximal end and a distal end, wherein the first section 25 is at the proximal end 21 of the sensor sheath 20 and the second section 27 is at the distal end 22 of the sensor sheath 20. An offset section 26 connects the distal end of the first section 25 and the proximal end of the second section 27 such that the first section 25 and the second section 27 are not co-linear.

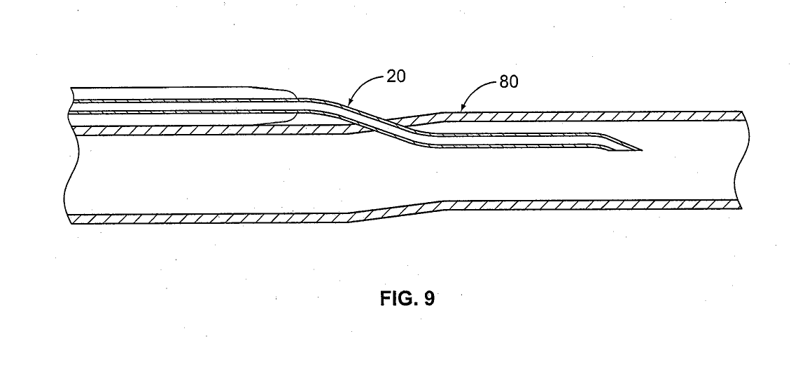

In another preferred embodiment, the optical device 10 may further comprise a rigid sensor body 50 covering a portion of the flexible optical fiber 30 adjacent to the proximal end 21 of the sensor sheath 20 that is not covered by the sensor sheath 20. FIG. 2C illustrates the optical sensing device 10 of FIG. 2B further having the sensor body 50. The first section 25 and the offset section 26 in the embodiments of FIGS. 2B and 2C may be used to establish the penetration depth close in proximity to the proximal end 21 of the sensor sheath 20 or the junction of the sensor sheath 20 and the rigid sensor body 50. The second section 27 may be used to elevate the distal end 22 of the sensor sheath 20 and the optical sensor 40 from the vascular wall to prevent walling issues by causing leverage about the penetration point lifting the sensor sheath 20 predictably against the penetration wall. In addition, this configuration of the sensor sheath 20 may increase the rate of fluid flow across the optical sensor 40, thereby cleaning the optical sensor 40 and preventing a buildup of static vascular components. FIG. 9 is an illustration of the embodiment of FIG. 2C showing the sensor body position with respect to the vascular wall 80.

The sensor sheath 20 of the optical sensing device 10 as illustrated in FIG. 2B or 2C may further be mechanically calibrated in length and depth to a particular position and angle with respect to the proximal end 21 of the sensor sheath 20 or the rigid sensor body 50 to allow an operator to reliably control its penetration properties.

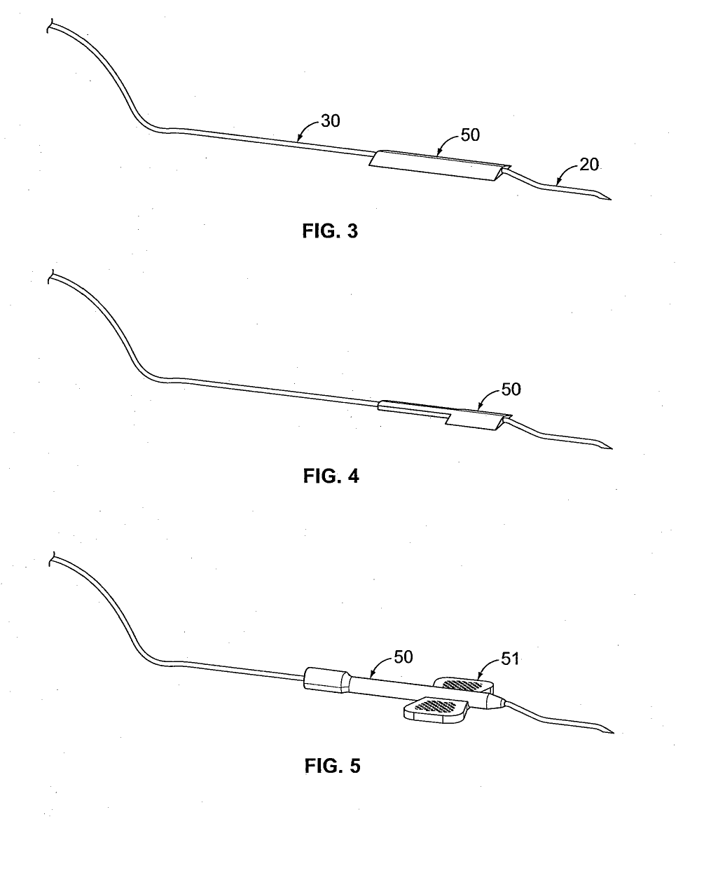

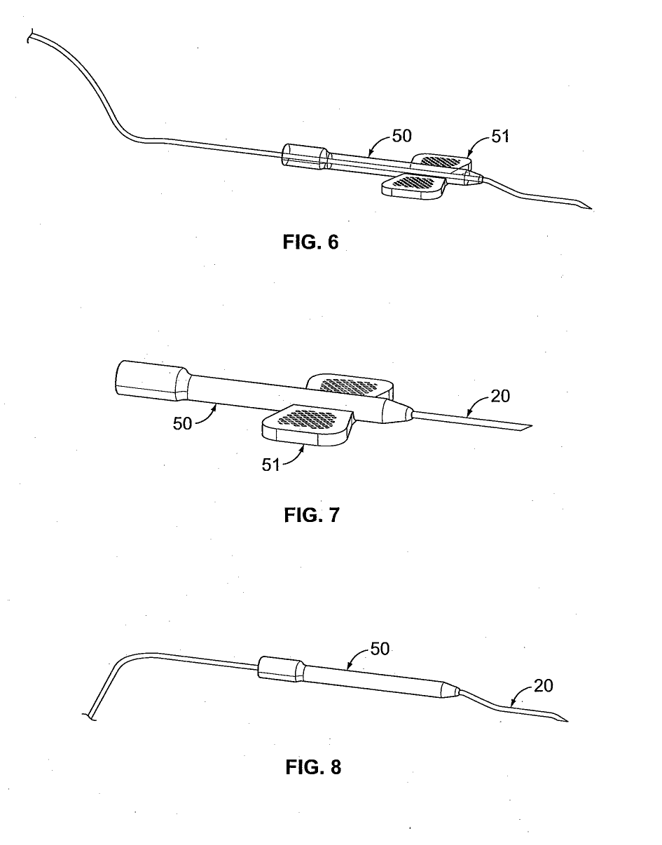

A portion of the proximal end 21 of the sensor sheath 20 as shown in FIG. 2A or 2B may be planar in shape. Similarly, in the embodiment with the rigid sensor body 50 as shown in FIG. 2C, the rigid sensor body 50 may be planer in shape (see FIGS. 3 and 4). The planar shape of the sensor body 50 may allow consistent calibrated alignment between the relative orientation of a base plane of the sensor body 50 and the sensor sheath 20. In addition, the sensor sheath 20 or the sensor body 50 may have one or more substantially planer protrusions 51 forming stabilization platforms. Such protrusions on a sensory body 50 are illustrated in FIGS. 4-7. The stabilization platforms further provide lateral and rotational stability to the sensor sheath 20, and maintain the desired position and orientation of the optical sensor 40 with respect to the vessel wall without continuous manual adjustment by the operator.

The sensor body 50 may be 2 to 4 cm in length, 0.5 to 4 cm in width, and 0.25 to 2 cm in height. Referring to FIGS. 4-8, the stabilization platforms may extend the width of the sensor body 50 by an additional 1 to 3 cm. In additional, stabilization platforms may be asymmetrically located closer to the sensor sheath 20 to provide the operator with greater control. Furthermore, the sensor body 50 and/or stabilization platforms 51 may be fixed with an adhesive, similar to adhesives used in conventional cutaneous bandages, allowing the operator to maintain proper device placement without the use of continuous manual effort. The sensor body 50 and planar stabilization protrusions 51 in one example may simply be combined in structure and function as one substantially planar structure, as shown in FIG. 3 and FIG. 4. The sensor body 50 may be injection modeled to surround the flexible optical fiber/sensor sheath junction with a medically acceptable material, such as poly ethylene. Materials such as poly ethylene and silicone may have properties sufficient to provide the necessary structural support for initial cutaneous puncturing and rotational stability, while providing the necessary flexibility to conform to the curvature of the cutaneous surface.

A second set of stabilization platforms may be formed around the flexible optical fiber 30 to allow for an adhesive surface to secure the flexible fiber to a separate location on the skin of a patient and prevent tension on the optical sensor 40 during use. This second set may have dimensions similar to the stabilization platforms on the sensor body.

The optical sensing device 10 may be manufactured in multiple steps using conventional techniques, such as mechanical pressing and injection molding. One suitable manufacturing process may comprise multiple steps beginning with a preformed hollow sensor sheath 20, such as a stainless steel tube. An optical fiber of a suitable material, such as glass or polymethylmethacrylate (PMMA), extending from a coated flexible fiber may then be mechanically inserted and secured within the sensor sheath 20, such that the distal end of the flexible optical fiber 30 is close in proximity to and aligns with the distal end of the sensor sheath 20. The sensor sheath/flexible fiber junction may then be surrounded in a sensor body 50 by using conventional methods, such as injection molding, of conventional materials, such as poly ethylene. The sensor sheath 20 may then be cut to the desired penetration length, such as 2 cm, bent to the desired angles and sharpened to a point, where the distal end of the flexible optical fiber 30 and the distal end 22 of the sensor sheath 20 are flush. Finally the optical fiber 30 may be polished using conventional techniques and covered with a lubricious coating. The finished optical sensing device may then be sterilized using conventional methods, such as exposure to ethylene oxide, and packaged in a sealed container.

This finished optical sensing device may then be used by a technician as follows. The technician may connect the proximal end of the flexible optical fiber to an optical detection device, such as an optical renal function analysis apparatus. The operator may then insert the sharpened end of the sensor sheath through the vascular wall and into the blood vessel, and place the sensor body in contact with the patient's skin causing the sensor sheath 20 to lift to puncture side of the vascular wall. Finally an adhesive either attached or separate from the sensor body 50 may be used to mechanically secure the sensor body 50 to the skin, allowing for continuous optical monitoring of vascular components without constant manual adjustment using a single puncturing device.

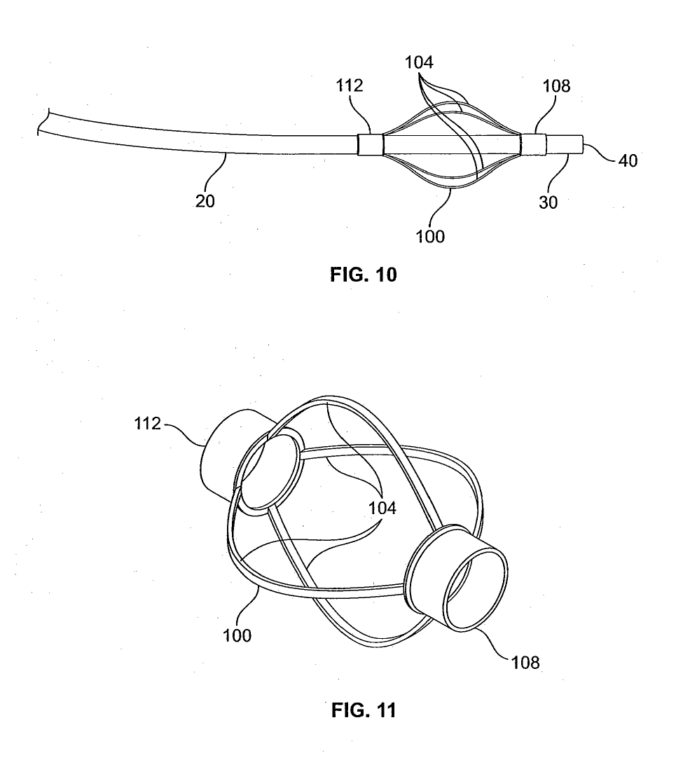

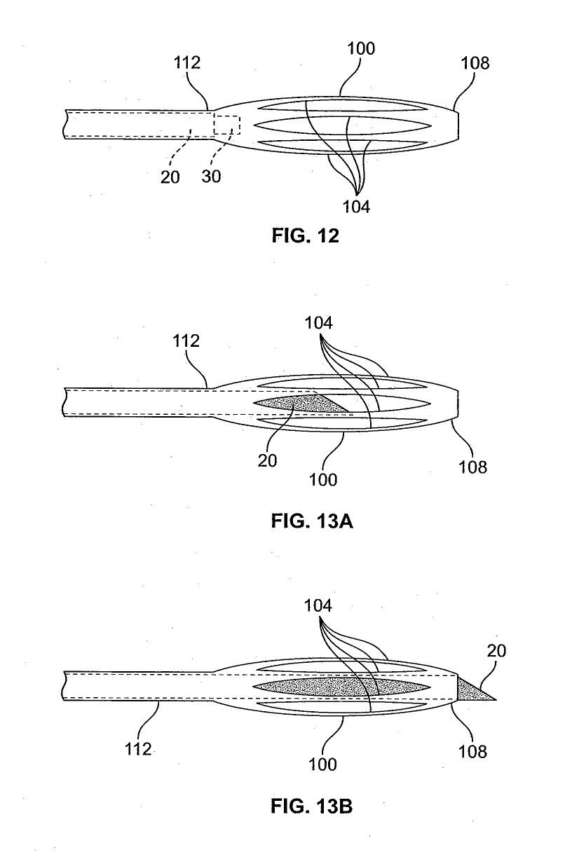

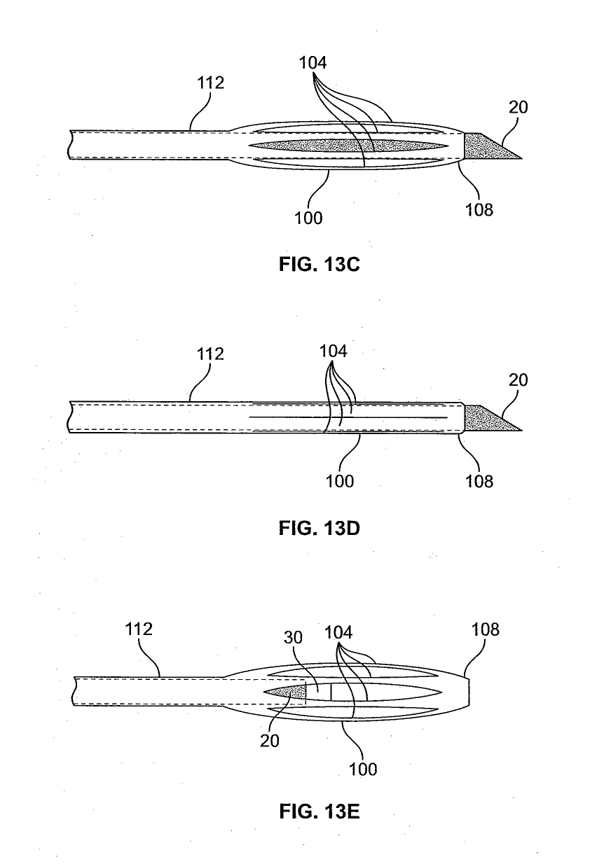

As illustrated in FIGS. 10-13, the optical sensing device may include an optical fiber 30 centering mechanism 100. The centering mechanism is located at the distal tip of the catheter and is expanded once the catheter is inserted into a blood vessel. The centering mechanism 100 is provided to center the tip of the optical fiber 30 in a flow of blood. This is helpful in reducing environmental noise attributable to fluorescence of surrounding anatomical structures such as vein walls.

The centering device includes a collapsible cage. The cage is preferably adapted to expand and contract upon an applied force. Accordingly, the cage may be produced from any material which has at least a partial memory in that it exhibits an elastic quality. In the preferred embodiment, the cage is formed from a plurality of spokes 104 arranged along a length of the optical sensing device. Embodiments having 3, 4, and 5 spokes 104 have been contemplated by the inventors.

The spokes 104 can be produced from a metallic material, such as an alloy of nickel and titanium. Each spoke 104 has a first terminal end generally fixed to an annular collar located at a first end 108 of the centering mechanism 100 and a second terminal end fixed to a second annular collar located at a second end 112 of the centering mechanism 100. The spokes 104 are capable of radially outwardly movement, in a bulging fashion, as relative movement between the collars brings the first end 108 closer to the second end 112. Stated another way, by decreasing a distance between the first and second ends 108, 112 upon relative movement of the collars, a portion of the diameter of the cage can be expanded to provide support structure against, for example, walls of a vein or other fluid carrying vessel. The portion of the diameter is collapsed upon increasing the distance between the collars.

The first end 108 of the centering mechanism 100 preferably forms a free point on the device, generally in capable of operable communication with a portion of the device, either via friction, adhesive or the like. The second end 112 is preferably attachable to the sensor sheath 20, such that it is primarily fixed. Some design notes, is that the cage must have enough strength to support itself off the bottom of the vein, so have worked with 3 spoke, 4 spoke and 5 spoke cages. The distal end 108 moves on the optical fiber 30 with the proximal end 112 stationary. The cage is packaged with a shroud that slides off when it enters the catheter to keep it from getting caught.

In use, the distal end 22 of the sheath 20 is inserted through a central portion of the cage between the spokes 104. A portion of the sheath 20 operatively engages the free end 108 of the centering mechanism 100. As the sheath extends through the cage from one end 112 to the other 108, a portion of the sheath 20 engages the centering mechanism and the centering mechanism collapses from its biased condition in an expanded state to a collapsed condition such that the centering mechanism 100 is capable of following the distal end of the sheath 20 into a vein or blood vessel. Once within the blood vessel, the distal end 22 of the sheath 20 is backed off a bit such that the centering mechanism 100 returns to its biased position in an expanded condition, and the fiber optic fiber 30 is thus within the central portion of the centering device 100, and the optical sensor 40 of the catheter is held centered within the flow of blood in, for example, a vein.

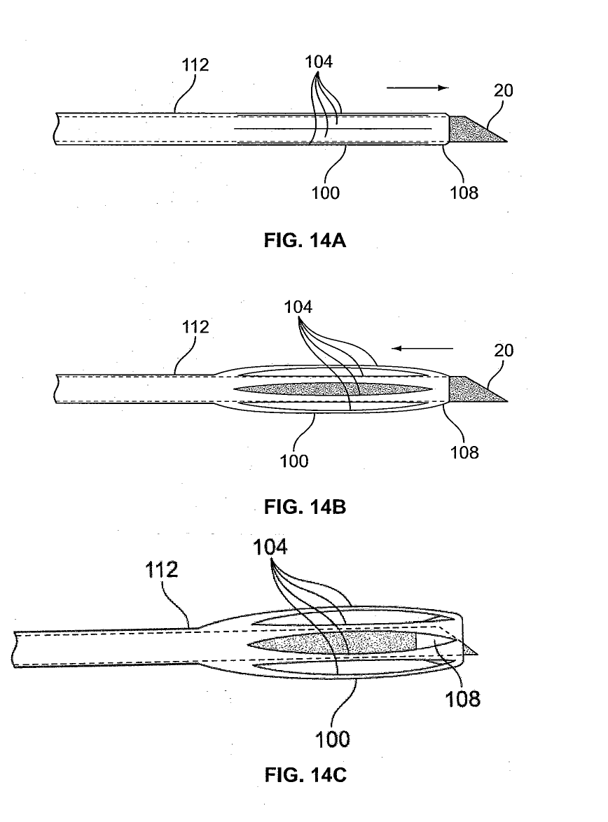

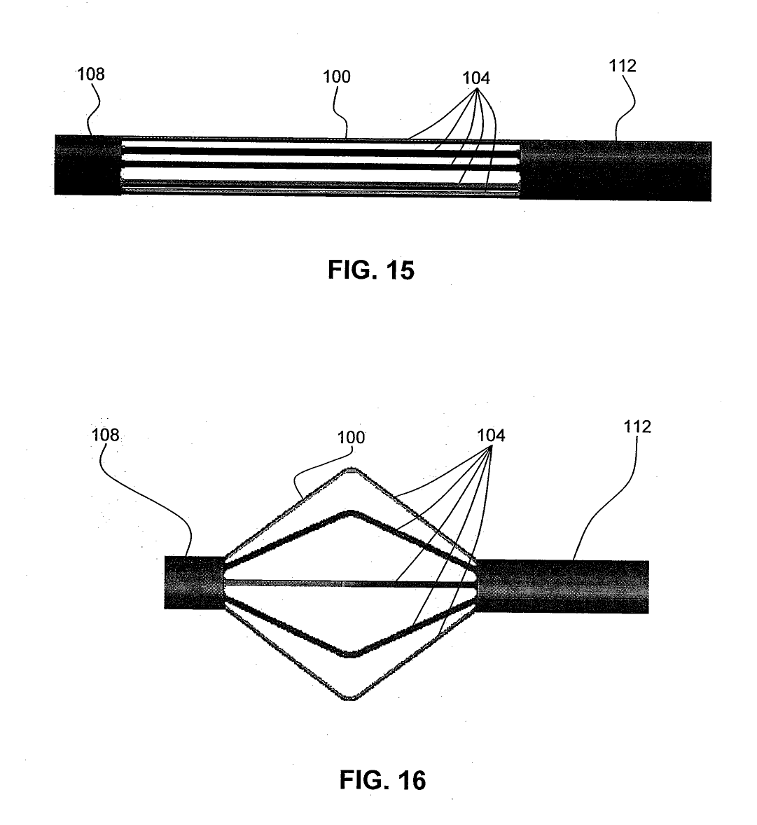

An optical device having an alternative centering device 100 is illustrated in FIGS. 14A through 14C. In this embodiment of the centering mechanism 100 the distal end 108 of the centering mechanism is attached, and at least substantially fixed, to the distal end 22 of the sensor sheath 20, preferably the distal end 108 of the centering mechanism 100 is bonded to the distal end 22 of sensor sheath 20. This allows remaining portions of the centering mechanism 100 and sensor sheath 20 to glide freely past each other. The spokes 104 of this embodiment, therefore, are expanded by manually pulling the sensor sheath 20 back while maintaining the position of the centering mechanism 100, as indicated by the directional arrows on the figures. This allows a relatively soft polymeric material with a very low restorative force to be used as the centering mechanism 100. When a sufficiently soft plastic is used, the distal end of the sensor sheath 20, including the optical fiber 30 and the optical sensor 40, is protected in the expanded position by allowing the soft material of the centering mechanism 100 to fold over itself as the sensor sheath 20 is retracted. (See FIG. 14C). A calibrated locking mechanism may maintain the relative position of the centering mechanism 100 and the sensor sheath 20 and tension in the retracted sheath 20 without continuous human intervention.

In use, the sharpened end 22 of the sheath 20 is used to puncture. The distal end 22 of the sheath 20 carries or transports the centering mechanism 100 into the fluid. The sheath 20 is then backed off or pulled slightly in the direction of the arrow in FIG. 14B. This retraction causes the spokes 104 to expand radially outwardly relative to the sheath 20. Further retraction, as illustrated in FIG. 14C, causes the distal end 108 of the centering mechanism 100 to enter the central portion of the centering mechanism 100 to further protect the optical sensor 40. It follows that still further retraction would cause the distal end 22 of the sheath 20 to fully enter the central portion of the centering mechanism 100 located radially inwardly of the spokes 104.

The optical sensor 40 may also gain access to the vascular fluid via a side opening in the sensor sheath 20 within the centering mechanism 100.

The terms “first,” “second,” “upper,” “lower,” “front,” “back,” “top,” “bottom,” etc. are used for illustrative purposes only and are not intended to limit the embodiments in any way. The term “plurality” as used herein is intended to indicate any number greater than one, either disjunctively or conjunctively as necessary, up to an infinite number. The terms “joined” and “connected” as used herein are intended to put or bring two elements together so as to form a unit, and any number of elements, devices, fasteners, etc. may be provided between the joined or connected elements unless otherwise specified by the use of the term “directly” and supported by the drawings. The term “elastic” as used herein is intended to indicate a form of material deformation wherein the structure or material has a measure of memory such that substantially returns to its original shape once a force responsible for the deformation is removed, i.e. change in shape of a material at a given stress is recoverable after the stress is removed. This differs from plastic deformation where permanent distortion of a material occurs under the action of an applied stress.

While the specific embodiments have been illustrated and described, numerous modifications come to mind without significantly departing from the spirit of the invention, and the scope of protection is only limited by the scope of the accompanying Claims.

What is claimed is:

1. A self-penetrating percutaneous optical sensing device for obtaining and transmitting optical signal from intravascular fluid in a blood vessel, the device comprising:
(a) an elongated hollow rigid sensor sheath having a proximal end, a distal end and a central channel extending along the sensor sheath, wherein the distal end of the sensor sheath is sufficiently sharpened to puncture a cutaneous barrier and the sensor sheath has a sufficient length to allow the sensor sheath to penetrate into intravascular space of a blood vessel;
(b) a flexible optical fiber having a proximal end and a distal end situated coherently within the central channel of the sensor sheath wherein the sensor sheath covers a portion of the distal end of the flexible optical fiber and wherein the distal end of the flexible optical fiber aligns with the distal end of the sensor sheath; and
(c) an optical sensor connected to the distal end of the flexible optical fiber wherein an optical signal generated at the optical sensor can be transmitted from the optical sensor to the proximal end of the flexible optical fiber via the flexible optical fiber and wherein the optical sensor has direct access to the intravascular fluid of the blood vessel.

2. The optical sensing device of claim 1 wherein the sensor sheath comprises a first section having a proximal end and a distal end and a second section having a proximal end and a distal end, wherein the first section is at the proximal end of the sensor sheath and the second section is at the distal end of the sensor sheath and wherein distal end of the first section and the proximal end of the second section is connected by an offset section such that the first section and the second section are not co-linear.

3. The optical sensing device of claim 2 wherein the sensor sheath is mechanically calibrated to a particular position and angle with respect to the first section of the sensor sheath.

4. The optical sensing device of claim 1 wherein a portion of the proximal end of the sensor sheath has one or more substantially planar protrusions to form stabilization platforms.

5. The optical sensing device of claim 1 further comprising a rigid sensor body covering a portion of the flexible optical fiber adjacent to the proximal end of the sensor sheath that is not covered by the sensor sheath.

6. The optical sensing device of claim 5 wherein the rigid sensor body is cylindrical or planar in shape.

7. The optical sensing device of claim 5 wherein the rigid sensor body has one or more substantially planar protrusions to form stabilization platforms.

8. The optical sensing device of claim 1 wherein the proximal end of the flexible optical fiber is connected to an optical detection device.

9. The optical sensing device of claim 5 wherein the optical sensor protrudes from the sensor sheath.

10. The optical sensing device of claim 1 wherein the optical sensor is coated with a material containing a chemically sensitive chromophore.

11. The optical sensing device of claim 1 further comprising a centering mechanism for centering the optical sensor within the intravascular fluid of the blood vessel.

12. A self-penetrating percutaneous optical sensing device for obtaining and transmitting optical signal from intravascular fluid in a blood vessel, the device comprising:
an elongated sheath having a proximal end, a distal end and a central channel extending along the sheath, wherein the sheath has a sufficient length to allow the sheath to penetrate into intravascular space of a blood vessel;
an optical fiber having a proximal end and a distal end situated coherently within the central channel of the sheath wherein the sheath covers a portion of the distal end of the optical fiber; and
a centering mechanism located at the distal end of the sheath having a diameter greater than a diameter of the distal end of the sheath.

13. The optical sensing device of claim 12 wherein the centering mechanism may be alternately placed in an expanded condition or a collapsed condition.

14. The optical sensing device of claim 13 wherein the centering can be selectively elastically deformed between the expanded condition and the collapsed condition upon application of an applied force.

15. The optical sensing device of claim 14 wherein the centering mechanism is provided to center a tip of the optical fiber in a flow of blood.

16. The optical sensing device of claim 15 wherein the centering device has a central passageway through which the sheath passes.

17. The optical sensing device of claim 16 wherein a portion of the sheath is operably engageable with a portion of the centering mechanism and wherein relative movement between the sheath and the centering mechanism causes the centering mechanism to change from the expanded condition to the collapsed condition.

18. The optical sensing device of claim 17 wherein the centering mechanism comprises a plurality of spokes wherein each spoke is fixed at opposing terminal ends to opposing portions of the centering mechanism.

19. The optical sensing device of claim 18 wherein the collapsed condition is generated by passing the sheath from a first end of the centering mechanism through the central passageway and exiting the centering mechanism at a second end of the centering mechanism.

20. The optical sensing device of claim 19 further comprising an optical sensor connected to the distal end of the optical fiber wherein an optical signal generated at the optical sensor can be transmitted from the optical sensor to the proximal end of the optical fiber via the optical fiber and wherein the optical sensor has direct access to the intravascular fluid of the blood vessel.

21. The optical sensing device of claim 12 wherein the centering mechanism comprises a first end segment separated from a second end segment by a midsection which expands radially outwardly upon relative movement between the first end segment and the second end segment which decreases a distance between the first and second end segments.

22. The optical sensing device of claim 12 wherein a distal end of the centering mechanism is in operative communication with the distal end of the sheath such that extension and retraction movements by the sheath are imparted to the distal end of the centering mechanism.

23. The optical sensing device of claim 22 wherein a portion of the centering mechanism expands radially outwardly relative to the sheath upon retraction of the distal end of the sheath towards the centering mechanism.

24. The optical sensing device of claim 23 wherein retraction of the distal end of the sheath causes the distal end of the centering mechanism to enter a central portion of the centering mechanism.

25. The optical sensing device of claim 24 wherein the distal end of the sheath has a sharpened end for percutaneous puncture which is at least partially located within the central portion of the centering mechanism upon retraction of the distal end of the sheath.

26. The optical sensing device of claim 25 wherein the central portion of the centering mechanism is located radially inwardly of a plurality of spokes joining opposite the distal end of the centering mechanism with an opposing proximal end of the centering mechanism.

27. The optical sensing device of claim 26 wherein the spokes expand radially outwardly upon retraction of the distal end of the sheath.
